(19) United States
(12) Patent Application Publication
(10) Pub. No.: US 2009/0112070 A1
(43) Pub. Date: Apr. 30, 2009

(54) Telemedicine Device and System

(76) Inventors: Yen-Shan LIN, Taipei City (TW); Han-Wei Zhang, Xindian City (TW)

Correspondence Address: DAVID I. ROCHE;BAKER & MCKENZIE LLP, 130 EAST RANDOLPH DRIVE, CHICAGO, IL, 60601, US

(21) Appl. No.: 12/262,416

(22) Filed: Oct. 31, 2008

(30) Foreign Application Priority Data
Oct. 31, 2007 (TW) 096141099

(51) Int. Cl. A61B 5/00; G06Q 50/00

(52) U.S. Cl. 600/300; 705/3

(57) ABSTRACT

The present invention is provided a telemedicine device and system, and the telemedicine device and system comprise at least a computer system and a channel-selecting device, wherein the computer system can connect with the internet and have at least one expanded module to connect with a plurality of periphery devices. The telemedicine system is combined with healthcare system, information system, equipment system and homecare system to put the government policies “aging in place” and “aging at home” into practice, and also provide the elders healthier, safer, more respectful, more comfortable and more convenient life qualities, and maintain the elders' health.

### PRIOR RELATED APPLICATIONS

This application claims priority to Taiwanese Patent Application Number 096141099 filed on Oct. 31, 2007 and incorporated herein by reference.

### FEDERALLY SPONSORED RESEARCH STATEMENT

Not applicable.

### REFERENCE TO MICROFICHE APPENDIX

Not applicable.

### FIELD OF THE INVENTION

The invention is broadly related to a telemedicine device and system and more particularly to a computer system with a channel-selecting device to measure the specific physiological information or to select the specific service and be connected with the specific system.

### BACKGROUND OF THE INVENTION

People average life raise year after year because of technology and medicine development. On the other hand, people health may be the worse for environment, diet and pressure, for example cardiovascular diseases, cancer, diabetes and chronic hepatitis. These civil and chronic diseases are almost higher ranking cause of death in the world.

The medical centers and medicalcare services nowadays are designed for immediate diseases. It is very insufficient for patients of chronic disease. In particular, symptom changes of chronic disease, psychological counseling and medicalcare matching and coordination. If chronic diseases are out of control, patients can not hospitalize right now. It further leads to waste of medical resource and patients can not receive a good attendance.

Thus, everyone should take care of the health by self using sport and diet. It is in good condition and reduces efficiently the medical cost of the government. In addition, the elders don't like to live at senior care centers in Chinese view. They generally like to live with their children or nearby. However, the children usually work busily and have no time to take good care of the elders. Particularly, some elders have a disability or movement inconvenience but no one can help them.

To establish the objects of “aging in place” and “aging at home” and further to provide the elders healthier, safer, more respectful, more comfortable and more convenient life qualities, and maintain the elders' health. It is necessary that a telemedicine device and system can be combined with medical centers, senior care centers and information service providers to advance the excellent and efficiency healthcare service of the elders.

### SUMMARY OF THE INVENTION

One object of the present invention is provided with a telemedicine device and system. The telemedicine device and system comprise at least a computer system, a first periphery device, a second periphery device and a channel-selecting device. The computer system of the present invention is connected to the Internet and has a first expanded module and a second expanded module, wherein the first expanded module and the second expanded module are connected to a display device. In addition, the first periphery device of the present invention can be connected to the computer system by the first expanded module to measure a first data of human body, and the first data are transmitted to the computer system. Furthermore, the second periphery device of the present invention can be connected to the computer system by the second expanded module to measure a second data of human body, and the second data are transmitted to the computer system. Moreover, the channel-selecting device of the present invention can transmit a selected signal to the computer system in order to switch on and off between the first data and the second data.

The telemedicine device of the present invention comprises at least the computer system. The computer system comprises at least one expanded module, wireless radio-frequency transmission modules, communication modules, wireless network modules, storage devices, and public switched telephone network (PSTN) modules. In addition, the display device comprises a display or a television. Furthermore, the periphery devices comprise at least a sphygmomanometer, a blood-glucose meter, an oximeter, an electrocardiograph, a bathroom scales, a thermometer, a peak expiratory flow meter, a rehabilitation device, a camera device, a record device, a mobile telephone and an Internet telephone. Moreover, the computer system of the present invention is connected to the Internet by wired or wireless way.

The channel-selecting device comprises a set of directional function key and a set of numerical input keyboard to select directly the channel. In addition, the channel-selecting device comprises a confirm button to start the operation of the selected channel, a cancel button to cancel the operation of the selected channel, and a back button to back the operation of the selected channel. Furthermore, the expanded modules can make use of universal serial bus (USB), bluetooth transmission, infrared transmission, peripheral component interconnect (PCI) bus, small computer system interface (SCSI) bus, personal computer memory card international association (PCMICA) slot or memory card slot.

Another object of the present invention is provided with a telemedicine system. The telemedicine system comprises at least a telemedicine device, an information integrated platform, a services matching system and a medicalcare system. The information integrated platform of the present invention is connected to the telemedicine device and receive the first data and the second data from said telemedicine device to get the integrated information. In addition, the services matching system of the present invention are connected to the information integrated platform and receive the integrated information to provide the daily living services. Furthermore, the medicalcare system of the present invention is connected to the information integrated platform and receives the integrated information to provide medicalcare services.

In addition, the daily living services of the services matching system comprise the service of healthy-food, healthy-clothing, healthy-housing, healthy-transportation, healthy-education and healthy-entertainment. Furthermore, the medicalcare services of the medicalcare system comprise management of physiological information, disposition of event happened, medicalcare matching and coordination, management of customer services, telemedicine services and evaluation of health-function.

The advantage of the present invention is provided with a telemedicine device and system. The telemedicine system is combined with healthcare system (for example medical centers or senior care centers), information system (for example information service providers or telecommunication service providers), equipment system (for example medical and healthcare devices) and homecare system to put the government policies “aging in place” and “aging at home” into practice. The telemedicine system also can provide the elders healthier, safer, more respectful, more comfortable and more convenient life qualities, and maintain the elders' health.

Further advantages and advantageous embodiments of the present invention are explained in more detail with reference to figures showing embodiments of the telemedicine device and system.

### BRIEF DESCRIPTION OF THE DRAWINGS

FIG. 1 shows an embodiment of a telemedicine device in accordance with the present invention.

FIG. 2 shows another embodiment of a telemedicine device in accordance with the present invention.

FIG. 3 shows an illustration of a computer system of the telemedicine device in accordance with the present invention.

FIG. 4 shows an illustration of a channel-selecting device in accordance with the present invention.

FIG. 5 shows an illustration of a telemedicine system in accordance with the present invention.

FIG. 6 shows an illustration of a services matching system of the telemedicine system in accordance with the present invention.

FIG. 7 shows an illustration of a medicalcare system of the telemedicine system in accordance with the present invention.

### DESCRIPTION OF EMBODIMENTS OF THE INVENTION

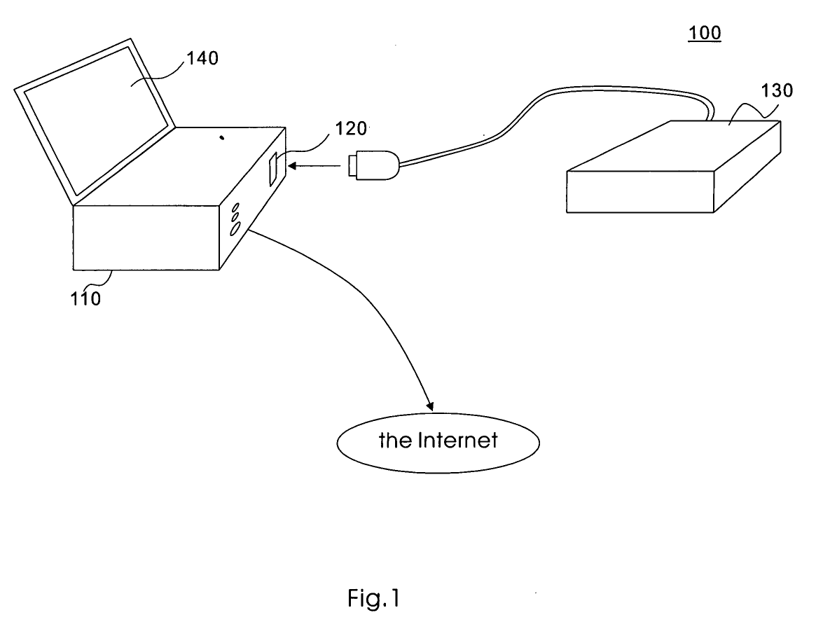

Reference is now made to FIG. 1, which is a simplified schematic diagram showing a first embodiment of a telemedicine device 100 in accordance with the present invention. The telemedicine device 100 comprises a computer system 110 that can be provided with at least one expanded module 120 and is connected to at least one periphery device 130. The touch screen device 140 can transmit a signal to the computer system 110 in work and shows related information. In addition, the computer system 110 is also connected to a display device 150 to show the information and data.

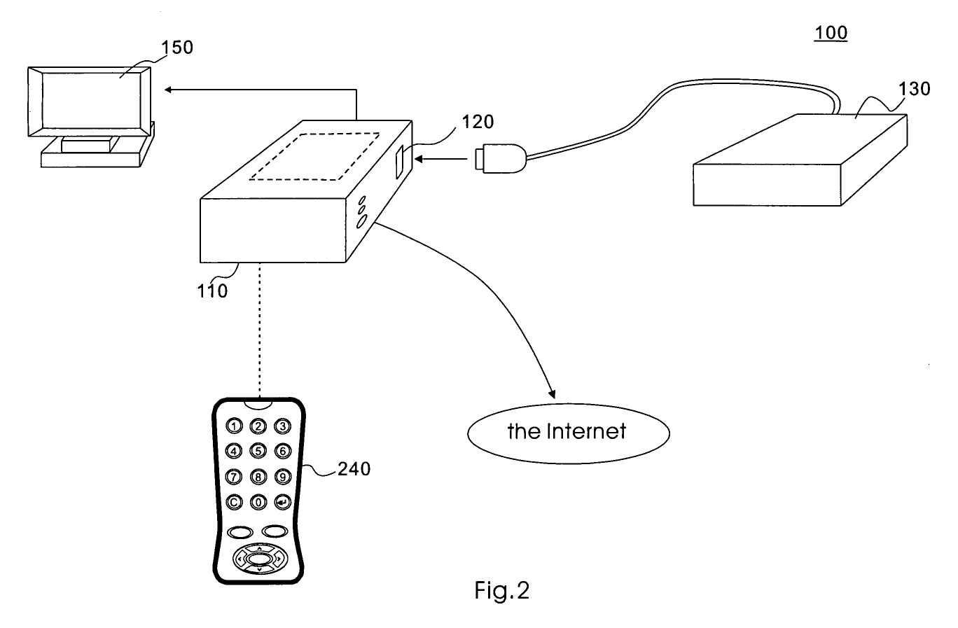

Reference is now made to FIG. 2, which is a simplified schematic diagram showing a first embodiment of a telemedicine device 100 in accordance with the present invention. The telemedicine device 100 comprises a computer system 110 that can be provided with at least one expanded module 120 and is connected to at least one periphery device 130. The touch screen device 140 can transmit a signal to the computer system 110 in work and shows related information. In addition, the computer system 110 is also connected to a display device 150 to show the information and data.

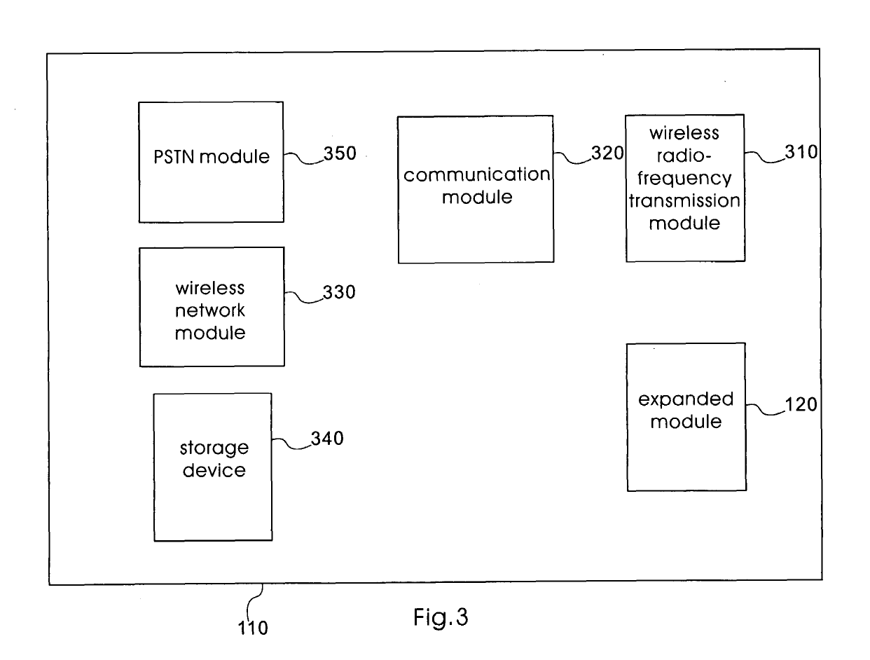

Reference is now made to FIG. 3, which is a simplified schematic diagram showing an embodiment of the computer system 110 in accordance with the present invention. The computer system 110 comprise at least one expanded module 120, a wireless radio-frequency transmission module 310, a communication module 320, a wireless network module 330, a storage device 340 and a public switched telephone network (PSTN) module 350. According to a preferred embodiment of the present invention, the expanded module 120 can make use of universal serial bus (USB), bluetooth transmission, infrared transmission, peripheral component interconnect (PCI) bus, small computer system interface (SCSI) bus, personal computer memory card international association (PCMICA) slot or memory card slot. In accordance with a preferred embodiment of the present invention, the wireless radio-frequency transmission module 210 can make use of radio-frequency transmission modules, wireless sensor network (WSN) transmission modules, ZigBee transmission modules, bluetooth transmission modules or ultra wide band (UWB) transmission modules. In accordance with another preferred embodiment of the present invention, the communication module 320 can make use of global system for mobile communications (GSM, 2G), universal mobile telecommunications system (UMTS, 3G) or high speed downlink packet access (HSDPA, 3.5G). According to another preferred embodiment of the present invention, the wireless network module 330 can make use of Wi-Fi modules or WiMax modules. Furthermore, according to another preferred embodiment of the present invention, the storage device 240 can make use of hard disks, disk on module (DOM) or flash memory.

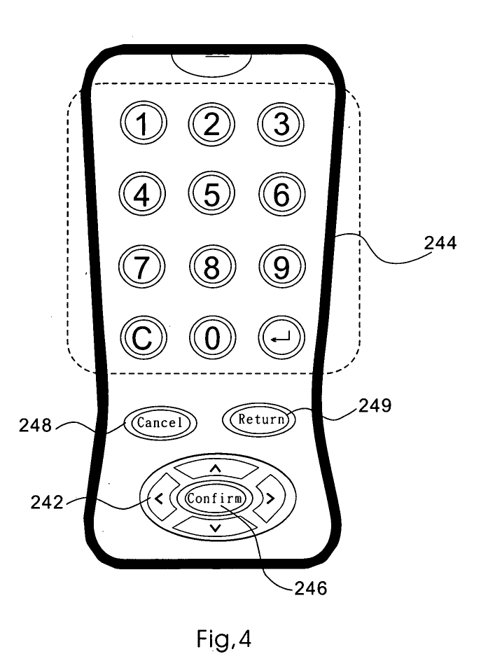

In FIG. 4, the channel-selecting device 240 comprises at least a set of directional function key 242 and a set of numerical input keyboard 244 to select directly the channel. The channel-selecting device 240 also comprises a confirm button 246 to start the operation of the selected channel, a cancel button 248 to cancel the operation of the selected channel and a back button 249 to back the operation of the selected channel.

For example, the periphery device 132 can be a sphygmomanometer, the periphery device 134 can be a bathroom scales, the periphery device 136 can be an internet telephone with a camera device, the periphery device 138 can be a camera device, and display device 150 can be a television. In addition, the expanded module 120 can make use of universal serial bus (USB), bluetooth transmission, infrared transmission, peripheral component interconnect (PCI) bus, small computer system interface (SCSI) bus, personal computer memory card international association (PCMICA) slot or memory card slot. Furthermore, the periphery device 132, 134 and 136 can be connected to the computer system 110 before the users use them. The information and data that the periphery devices measured, received or transmitted to the computer system 110 can be shown on the touch screen device 140 or the display device 150 at the same time.

In accordance with a preferred embodiment of the present invention, the user can use the computer system 110 to measure the blood pressure by means of the touch screen device 140 or the channel-selecting device 240.

The user should keep the posture to get an exact data with measuring by himself/herself, i.e. the height of the user's arm can not be higher then the height of the user's heart. The user want to use a sphygmomanometer 132, and then operates the periphery device 136 with the internet telephone of the camera device to work the computer system 110 for the selected the identifiable function of posture-scanning by means of the tough screen device 140 or the channel-selecting device 240. The selected identifiable function of posture-scanning is worked correctly after the user measures the blood pressure through using the directional function key 242 or the numerical input keyboard 244 of the channel-selecting device 240, and then the user presses the confirm button 246. The computer system 110 determines whether the user keeps the foregoing posture. If yes, he/she can start to measure the blood pressure by the sphygmomanometer 132.

During the foregoing process of the measuring posture, the picture or digital image taken by the camera device will be compared with the database of the computer system 110 to ensure whether they are similar enough in a tolerant range. If yes, the user can use the sphygmomanometer 132, but otherwise the display device 150 shows the notice of the incorrect posture as out of the tolerant range. The tolerant range is set for the optimum posture according to the manufacturer's measuring data, for example the height between the user's arm and the heart within 5 cm, and the angle between the user's upper arm and horizontal within 15°.

In accordance with another preferred embodiment of the present invention, the graphic notice of “the incorrect posture of measuring blood press” is shown on the computer system 110 when the computer system 110 determined that the posture of the user is incorrect and hence the user can not use the sphygmomanometer 132. The selected identifiable function of posture-scanning is worked correctly after the user measures the blood pressure again through using the directional function key 242 or the numerical input keyboard 244 of the channel-selecting device 240, and then the user presses the confirm button 246.

The user operates a sphygmomanometer 132 and views the information shown on the touch screen device 140 or the display device 150 firstly, and then the user measures the blood pressure through using the directional function key 242 or the numerical input keyboard 244 of the channel-selecting device 240. The measured data can be transmitted from the sphygmomanometer 132 to the computer system 110 and be shown on the touch screen device 140 or the display device 150 after the user presses the confirm button 246. However, the user also can press the cancel button 248 to cancel the operation of the selected channel or press the back button 249 to back the operation of the selected channel.

In accordance with another preferred embodiment of the present invention, the user can use the computer system 110 to measure the body weight by means of the touch screen device 140 or the channel-selecting device 240. The user operates a bathroom scale 134 and views the information shown on the display device 150 firstly, and then the user measures the body weight through using the directional function key 242 or the numerical input keyboard 244 of the channel-selecting device 240. The user also measures the body weight through using the touch screen device 140. The measured data can be transmitted from the bathroom scales 134 to the computer system 110 and be shown on the touch screen device 140 or the display device 150 after the user presses the confirm button 246. However, the user also can press the cancel button 248 to cancel the operation of the selected channel or press the back button 249 to back the operation of the selected channel.

According to one embodiment of the present invention, the periphery device 130 comprises at least a sphygmomanometer and a bathroom scales. The periphery device 130 can further comprise the devices that can be measured physiological information, for example a blood-glucose meter, an oximeter, an electrocardiograph, a thermometer, a peak expiratory flow meter or a rehabilitation device.

Furthermore, according to another embodiment of the present invention, the periphery device 130 also can comprise other devices, for example a camera device, a record device, a mobile telephone or an internet telephone.

Besides the channel-selecting device 240, the computer system 110 can be connected to the Internet and other users on the Internet by wired or wireless way.

As described above, the present invention in particular will enable elders, children, the users who can not operate the computer or the users who have a dislike for the computer to enjoy the convenience from the computer technology. Therefore, according to a preferred embodiment of the present invention, the periphery device 136 can be an Internet telephone with a camera device. The user can use the computer system 110 to operate the Internet telephone 136 by means of the touch screen device 140 or the channel-selecting device 240. The user views the information shown on the display device 150 and operates the internet telephone 136 through using the directional function key 242 or the numerical input keyboard 244 of the channel-selecting device 240. The Internet telephone 136 can be used after the user presses the confirm button 246. However, the user also can press the cancel button 248 to cancel the operation of the selected channel or press the back button 249 to back the operation of the selected channel. In addition, the camera device 138 can be used to take the daily life on a video file. The video file can use the computer system 110 through using the touch screen device 140 or the channel-selecting device 240, and can be transferred to the Internet, for example the personal webpage or the blog. The present invention can enable elders, children, the users who can not operate the computer or the users who have a dislike for the computer to feel the technological convenience by the telemedicine device 100.

In accordance with another preferred embodiment of the present invention, the user want to login the computer system 110 and show his/her personal data, and then operates the periphery device 136 with the internet telephone of the camera device to work the computer system 110 for the selected identifiable function of face-scanning by means of the tough screen device 140 or the channel-selecting device 240. The identifiable function of face-scanning is worked correctly after the user uses it through using the directional function key 242 or the numerical input keyboard 244 of the channel-selecting device 240, and then the user presses the confirm button 246. The picture or digital image taken by the camera device will be compared with the database of the computer system 110 to find the user's personal data. Finally, the user can login the computer system 110 and use the periphery devices to measure physiological information, for example a blood-glucose meter, an oximeter, an electrocardiograph, a thermometer, a peak expiratory flow meter or a rehabilitation device.

In accordance with another preferred embodiment of the present invention, the graphic notice of “no matched personal data” is shown on the computer system 110 when the computer system 110 does not find matched personal data. The selected identifiable function of face-scanning is worked correctly after the user try to login the computer system 110 again through using the directional function key 242 or the numerical input keyboard 244 of the channel-selecting device 240, and then the user presses the confirm button 246.

Besides, the user also can select the function of “add a new user” by the channel-selecting device 240, i.e. he/she takes a picture of digital image by the camera device and save them into the database of the computer system 110.

In accordance with one preferred embodiment of the present invention, the picture or digital image taken by the camera device will be compared with the database of the computer system 110 during the foregoing process of the identifiable function of face-scanning, for example it is determined respectively for the outline of face, eyes, nose, lip or mouth.

In accordance with another embodiment of the present invention, the database saved the personal data also can be mounted at a remote server of medical centers, i.e. the picture or digital image taken by the camera device are transmitted to the remote server at the medical centers by the Internet, and then the matched personal data is transmitted back to the computer system 110 from the remote server of the medical centers by the Internet. The user can login accordingly and use the periphery devices to measure physiological information.

The periphery devices 132, 134 and 136 were connected with the computer system 110 when the user operates them. The information which was measured, received or transmitted by the periphery devices is transmitted to the computer system 110 and be shown on the display device 150 or the touch screen device 140.

Thus, the users can select easily the channel like a television to operate the internet telephone without complex steps for starting the computer and can be in communication with friends, relatives and medicalcare centers cheaply. The users even don't need to learn how to connect with the internet or how to start the programs. The users also can operate other function of the periphery devices, for example a sphygmomanometer, a blood-glucose meter, an oximeter, an electrocardiograph, a bathroom scales, a thermometer, a peak expiratory flow meter and a rehabilitation device.

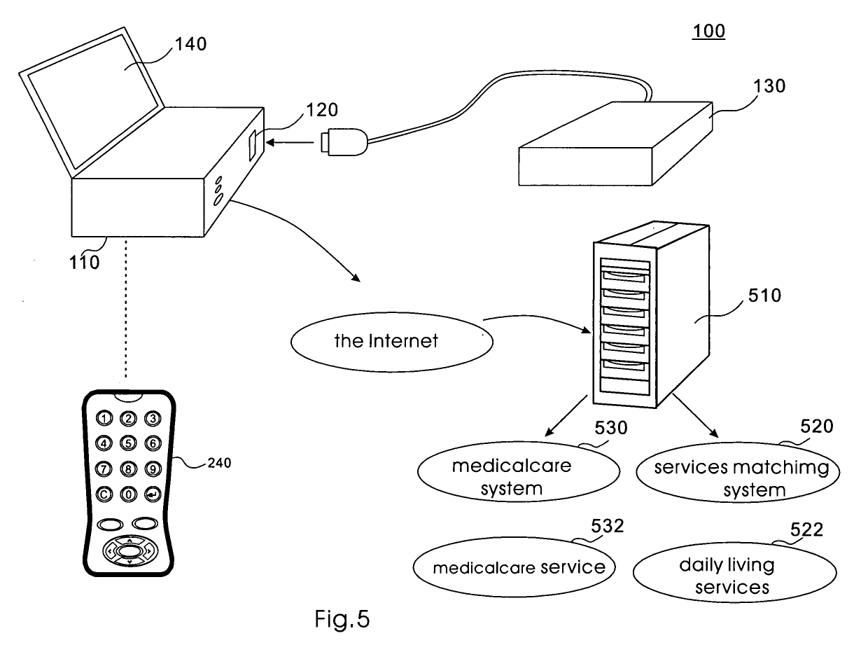

Reference is now made to FIG. 5, which is a simplified schematic diagram showing one embodiment of a telemedicine system 400 in accordance with the present invention. The telemedicine system 400 comprises the telemedicine device 100, an information integrated platform 510, a services matching system 520 and a medicalcare system 530. The information integrated platform 510 can be connected to the telemedicine device 100 and receives the information and data from the telemedicine device 100 to get the integrated information. In addition, the services matching system 520 can be connected to the information integrated platform 510 and receives the integrated information to provide the daily living services 522. Furthermore, the medicalcare system 530 can be connected to the information integrated platform 510 and receives the integrated information to provide the medicalcare service 532.

In accordance with a preferred embodiment of the present invention, the user can use the computer system 110 to make use of the services matching system 420 by means of the touch screen device 140 or the channel-selecting device 240. The user operates the services matching system 420 and views the information shown on the touch screen device 140 or the display device 150 through using the directional function key 242 or the numerical input keyboard 244 of the channel-selecting device 240. For example, the user can choose the service of healthy-entertainment 602 and press the confirm button 246. This order can be transmitted from the computer system 110 to the information integrated platform 510 via the Internet. The information integrated platform 510 can receive the orders from the user such as art appreciation and console game downloading and then can transmit the orders to daily living services 522 in work.

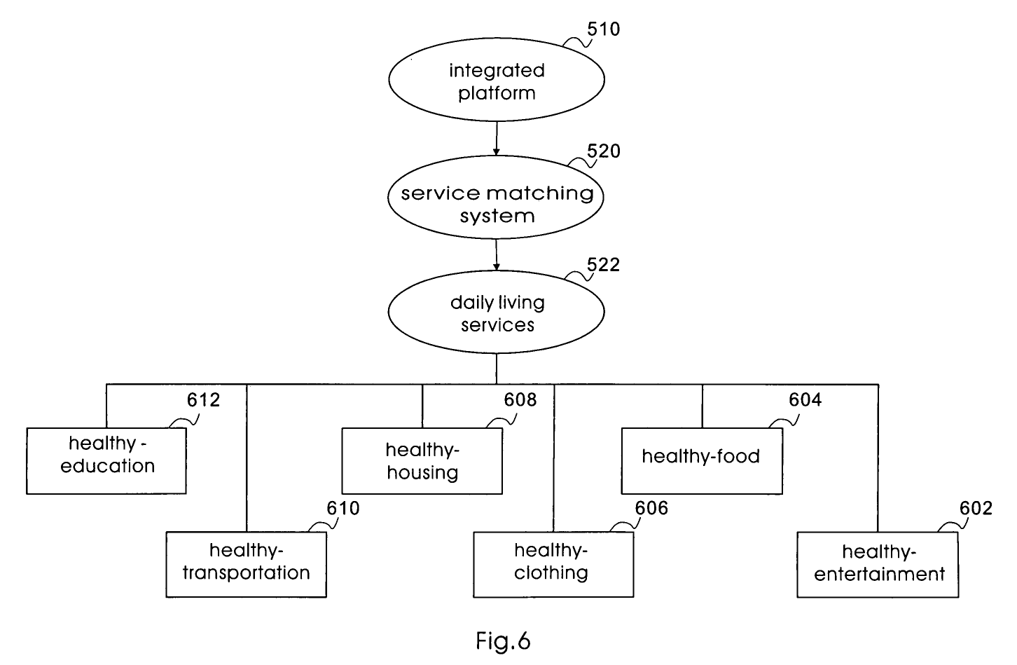

Reference is now made to FIG. 6, which is a simplified schematic diagram showing an embodiment of the daily living services 522 of the services matching system 520 in accordance with the present invention. Besides the service of the healthy-entertainment 602, the daily living services 522 further comprises the services of healthy-food 604, healthy-clothing 606, healthy-housing 608, healthy-transportation 610 and healthy-education 612. The services of the healthy-food 604 comprise the express services of healthy-meal or living goods. In addition, the services of the healthy-clothing 606 comprise the services of clothing washing or the clothing suggestion depending on the weather. Furthermore, the services of the healthy-housing 608 comprise the escort or traffic services of the outing. Moreover, the services of the healthy-transportation comprise the services of the living and health information supplying. Thus, one object of the present invention is provided with the excellent and convenient services by means of simple operation to satisfy the daily requirements for the users.

In accordance with another preferred embodiment of the present invention, the user can use the computer system 110 to make use of the medicalcare system 530 by means of the touch screen device 140 or the channel-selecting device 240. The user operates the medicalcare system 530 and views the information shown on the screen 140 or the display device 150 through using the directional function key 242 or the numerical input keyboard 244 of the channel-selecting device 240. For example, the user can choose the service of disposition of event happened 704 and press the confirm button 246. This order can be transmitted from the computer system 110 to the information integrated platform 510 via the Internet. The information integrated platform 510 can receive the orders from the user such as the notice when the measured physiological data is unusual, sending the reminder of news in brief and sending the reminder of email, and then can transmit the orders to the medicalcare service 532 in work. At the medicalcare services 532, the information integrated platform 510 can give the user notice via the computer system 110 when the user chose the service of disposition of event happened 704 and the measured physiological data is unusual. For example, the information integrated platform 510 can send the reminder of news in brief or the reminder of email to user's relatives and medicalcare centers. Thus people-related can control all the healthy status of the user and treat in an emergency.

According to another preferred embodiment of the present invention, the user can use the computer system 110 to make use of the services of the medicalcare matching and coordination 706 of the medicalcare service 532 by means of the touch screen device 140 or the channel-selecting device 240. The user operates the services of the medicalcare matching and coordination 706 and views the information shown on the touch screen device 140 or the display device 150 through using the directional function key 242 or the numerical input keyboard 244 of the channel-selecting device 240. For example, the user can choose the medicalcare matching and coordination 706 and press the confirm button 246. This order can be transmitted from the computer system 110 to the information integrated platform 510 via the Internet. The information integrated platform 510 can receive the orders from the user such as medicalcare calendars, integration and management of physiological information, matching with volunteers and medicalcare staffs and coordination system for a plurality of topic, and then can transmit the orders to the medicalcare services 532 in work. The medicalcare services 532 can further provide the services user needs, for example rehabilitation and checkup, by the medicalcare calendars or the physiological information, when the user chose the service of medicalcare matching and coordination 706.

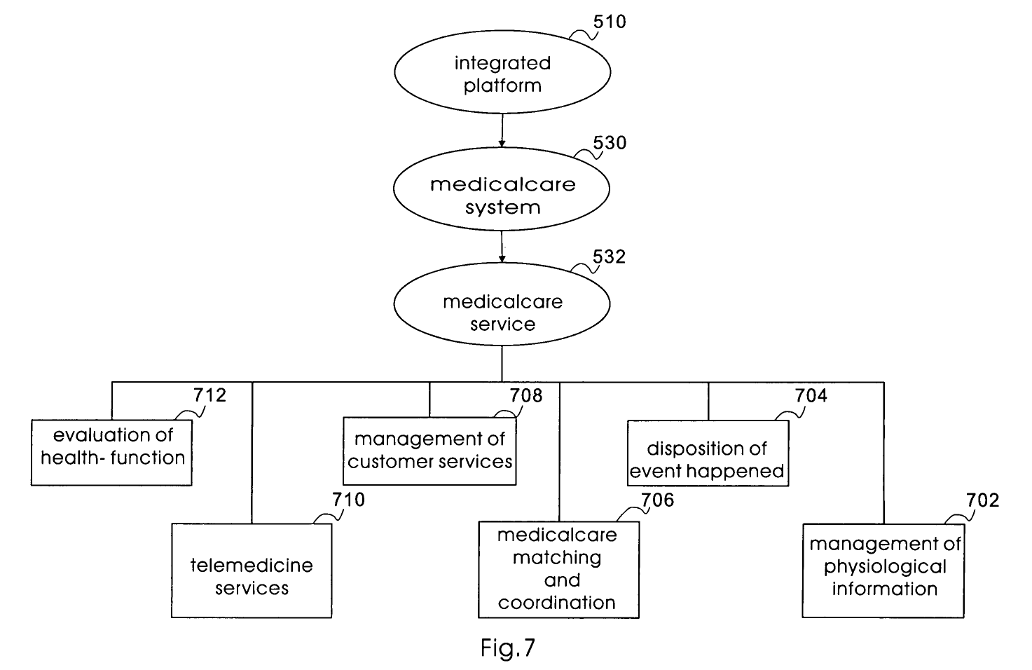

According to another preferred embodiment of the present invention, reference is now made to FIG. 7, the medicalcare services 532 are selected from the group consisting of management of physiological information 702, disposition of event happened 704, medicalcare matching and coordination 706, management of customer services 708, telemedicine services 710 and evaluation of health-function 712.

In an embodiment according to the present invention, the management of physiological information comprises the collection of physiological information, the analysis by diagram and the setting of physiological-limit-data. In another embodiment according to the present invention, the disposition of event happened comprises the notice when the measured physiological data is unusual, sending the reminder of news in brief and sending the reminder of email. In a preferred embodiment according to the present invention, the medicalcare matching and coordination comprises medicalcare calendars, integration and management of physiological information, matching with volunteers and medicalcare staffs and coordination system for a plurality of topic. In another preferred embodiment according to the present invention, the management of customer services further comprises the dispatching of volunteers, in-house visiting services, and hospitalization in an emergency, managements about each case and reports about any message. In another preferred embodiment according to the present invention, the telemedicine services comprise taking physiological information, communication and coordination of medicalcare services, and assistance of self-management. In another preferred embodiment according to the present invention, the evaluation of health-function comprises re-examination of health-function, screening of dementia, screening of nutritional status and screening of vital function.

One object of the present invention can be provided with the skin of humanization by design, in particular can enable elders, children, the user can not operate the computer or the user had a dislike for the computer to measure the specific physiological information or to select the specific services by the channel-selecting device 240. Furthermore, the telemedicine device is selected from the group consisting of identity-input mode, card-identify mode, graphic signal mode, face-scanning identify mode and auto-update services to tell apart a plurality of users and set up the personal data of users. At the same time, the telemedicine device can avoid an inconvenience because of the complex operation. Moreover, the telemedicine device is also provided with auto-update services from the information integrated platform 510 by the Internet.

The principles and operation of a telemedicine device and system according to the present invention may be better understood with reference to the drawings, embodiments and accompanying description.

The present invention is not restricted by the description on the basis of the embodiments. Rather, the present invention encompasses any new feature and also any combination of features, even if this feature or this combination itself is not explicitly specified in the patent claims or embodiments.

What is claimed is:

1. telemedicine device, comprises:
a computer system, which is connected to the Internet and is provided with a first expanded module and a second expanded module, wherein the first expanded module and the second expanded module are connected to a display device;
a first periphery device, which is connected to said computer system by the first expanded module to measure a first data of human body, wherein the first data are transmitted to said computer system;
a second periphery device, which is connected to said computer system by the second expanded module to measure a second data of human body, wherein the second data are transmitted to the computer system; and
a touch screen device, which is applied to show said first date and said second date and to transmit a selected signal to said computer system in order to switch on and off between the first data and the second data.

2. The device of claim 1, wherein said computer system further comprises wireless radio-frequency transmission modules, communication modules, wireless network modules, storage devices, and public switched telephone network (PSTN) modules.

3. The device of claim 2, wherein the wireless radio-frequency transmission modules are selected from the group consisting of radio-frequency transmission modules, wireless sensor network (WSN) transmission modules, ZigBee transmission modules, bluetooth transmission modules, and ultra wide band (UWB) transmission modules.

4. The device of claim 2, wherein the communication modules are selected from the group consisting of global system for mobile communications (GSM, 2G), universal mobile telecommunications system (UMTS, 3G), and high speed downlink packet access (HSDPA, 3.5G).

5. The device of claim 2, wherein the wireless network modules are selected from the group consisting of Wi-Fi modules and WiMax modules.

6. The device of claim 1, wherein the expanded modules are selected from the group consisting of universal serial bus (USB), bluetooth transmission, infrared transmission, peripheral component interconnect (PCI) bus, small computer system interface (SCSI) bus, personal computer memory card international association (PCMICA) slot, and memory card slot.

7. The device of claim 1, wherein the periphery devices are selected from the group consisting of a sphygmomanometer, a blood-glucose meter, an oximeter, an electrocardiograph, a bathroom scales, a thermometer, a peak expiratory flow meter, a rehabilitation device, a camera device, a record device, a mobile telephone and an internet telephone.

8. The device of claim 7, wherein the camera device has the identifiable function of face-scanning to tell apart a plurality of the personal data of users.

9. The device of claim 7, wherein the camera device has the identifiable function of posture-scanning to operate a sphygmomanometer and measure the blood pressure.

10. A telemedicine device, comprises:
a computer system, which is connected to the Internet and is provided with a first expanded module and a second expanded module, wherein the first expanded module and the second expanded module are connected to a display device;
a first periphery device, which is connected to said computer system by the first expanded module to measure a first data of human body, wherein the first data are transmitted to said computer system;
a second periphery device, which is connected to said computer system by the second expanded module to measure a second data of human body, wherein the second data are transmitted to the computer system; and
a channel-selecting device, which is applied to transmit a selected signal to said computer system in order to switch on and off between the first data and the second data.

11. The device of claim 10, wherein said computer system further comprises wireless radio-frequency transmission modules, communication modules, wireless network modules, storage devices, and public switched telephone network (PSTN) modules.

12. The device of claim 11, wherein the wireless radio-frequency transmission modules are selected from the group consisting of radio-frequency transmission modules, wireless sensor network (WSN) transmission modules, ZigBee transmission modules, bluetooth transmission modules, and ultra wide band (UWB) transmission modules.

13. The device of claim 11, wherein the communication modules are selected from the group consisting of global system for mobile communications (GSM, 2G), universal mobile telecommunications system (UMTS, 3G), and high speed downlink packet access (HSDPA, 3.5G).

14. The device of claim 11, wherein the wireless network modules are selected from the group consisting of Wi-Fi modules and WiMax modules.

15. The device of claim 10, wherein the expanded modules are selected from the group consisting of universal serial bus (USB), bluetooth transmission, infrared transmission, peripheral component interconnect (PCI) bus, small computer system interface (SCSI) bus, personal computer memory card international association (PCMICA) slot, and memory card slot.

16. The device of claim 10, wherein the periphery devices are selected from the group consisting of a sphygmomanometer, a blood-glucose meter, an oximeter, an electrocardiograph, a bathroom scales, a thermometer, a peak expiratory flow meter, a rehabilitation device, a camera device, a record device, a mobile telephone and an internet telephone.

17. The device of claim 16, wherein the camera device has the identifiable function of face-scanning to tell apart a plurality of the personal data of users.

18. The device of claim 16, wherein the camera device has the identifiable function of posture-scanning to operate a sphygmomanometer and measure the blood pressure.

19. A telemedicine system comprises:
a telemedicine device, which is comprised of at least a computer system which is connected to the Internet and is provided with a first expanded module and a second expanded module, wherein the first expanded module and the second expanded module are connected to a display device; a first periphery device which is connected to said computer system by the first expanded module to measure a first data of human body, wherein the first data are transmitted to said computer system; a second periphery device which is connected to said computer system by the second expanded module to measure a second data of human body, wherein the second data are transmitted to the computer system; and a channel-selecting device which is applied to transmit a selected signal to said computer system in order to switch on and off between the first data and the second data;
an information integrated platform, which is connected to said telemedicine device to receive the first data and the second data from said telemedicine device to get the integrated information;
a services matching system, which is connected to said information integrated platform to receive the integrated information to provide daily living services; and
a medicalcare system, which is connected to said information integrated platform to receive the integrated information to provide medicalcare services.

20. The system of claim 18, wherein said telemedicine device is selected from the group consisting of identity-input mode, card-identify mode and auto-update services to tell apart a plurality of users and to set up the personal data of users.

21. The system of claim 18, wherein the medicalcare services are selected from the group of management of physiological information, disposition of event happened, medicalcare matching and coordination, management of customer services, telemedicine services and evaluation of health-function.
